(19) United States
(12) Patent Application Publication
(10) Pub. No.: US 2023/0018232 A1
(43) Pub. Date: Jan. 19, 2023

(54) Search Engine to Provide Output Related to Bioinformatic Markers

(71) Applicant: MetaParadigm Wealth Management LLC, Staten Island, NY (US)

(72) Inventor: Salvatore Picciallo, Staten Island, NY (US)

(21) Appl. No.: 17/865,193

(22) Filed: Jul. 14, 2022

Related U.S. Application Data
(60) Provisional application No. 63/221,922, filed on Jul. 14, 2021.

(51) Int. Cl. G16H 50/20; G16H 50/30; G16H 50/70; G16H 20/10

(52) U.S. Cl. CPC G16H 50/20; G16H 20/10; G16H 50/30; G16H 50/70

(57) ABSTRACT

Technology is described for searching for treatment related output defined by bioinformatic markers of a person. The method can include receiving a biologic risk profile for the person which defines a plurality of disease risks expressed in bioinformatic markers. A disease interest profile representing diseases in which the person has interests may also be received. Disease keywords may be retrieved from the biologic risk profile and the disease interest profile. The disease keywords may be weighted using the disease interest profile. Further, the disease keywords may be mapped to medical publications, medical trials, or medical treatments to form a medical mapping. Disease keywords with a weighting above a defined weight threshold may be selected and disease keywords below the defined weight threshold may be filtered out. An asset that is associated with the disease keywords may be identified using a machine learning model to process the disease keywords and weightings.

### PRIORITY DATA

This application claims the benefit of United States Provisional Patent Application Serial No. 63/221,922, filed on Jul. 14, 2021 which is incorporated herein by reference.

### BACKGROUND

Biological testing for medical conditions has continued to advance in recent years. The number and variety of tests have grown, along with improvements in treatments for diseases identified by such tests. The ability to detect and identify medical, genetic, proteomics, functional or genomic risks for a person’s health has advanced dramatically in recent years. A number of genetic tests, genomic tests and other medical tests exist to identify whether a person has or is predisposed to certain diseases.

Once an individual identifies a predisposition for or probability for disease that exists in their own personal genetics or genome, the individual may be interested in identifying resources such as treatments, doctors, non-profits, companies and/or organizations to assist the individual. These resources may be used to support and treat individuals or families with diseases, conditions or biological pre-dispositions to diseases. However, identifying which treatments and organizations are effective in assisting those with genetic or other pre-dispositions to diseases can be difficult. In fact, some organizations which could be helpful to an individual may still be in the early research stages of working on a disease treatment. When an individual with identified genetic or other medical predispositions can identify treatments, medical professionals or organizations which are effectively researching, working on, testing or treating the diseases, conditions or disease risks that a person has, then the individual may be able to organize the individual’s resources to be involved with the treatments, doctors, companies, organizations, or non-profits who may assist them.

### BRIEF DESCRIPTION OF THE DRAWINGS

FIG. 1A is a block diagram illustrating an example of a system for searching medical data stores using bioinformatic markers of a person.

FIG. 1B is a block diagram illustrating an example of a system for searching medical data stores using bioinformatic markers of a person and using a purchase process.

FIG. 2 is an example of a flowchart illustrating a method of identifying a furthest developmental effort for treatment of diseases using the bioinformatic markers of a person.

FIG. 3 is a block diagram illustrating an example of a method for using bioinformatic markers of a person with a matrix or index to identify treatment related output or output records with a defined amount of risk from medical data stores.

FIG. 4 is a block diagram illustrating an example of a system for using hemophilia bioinformatic markers of a person to search for treatment related output or output records from medical data stores.

FIG. 5 is flowchart illustrating an example of a method for searching for treatment output records from medical data stores using bioinformatic markers of a person.

FIG. 6 is a block diagram illustrating an example of a service provider environment (e.g., a public or private cloud) upon which machine learning models or other search engines for this technology may execute.

FIG. 7 is a block diagram illustrating an example of computer hardware upon which this technology may execute.

### DETAILED DESCRIPTION

A technology is described that includes a system and method for using the bioinformatic markers of a person to search for treatment related output or treatment related output records. The treatment related output provided may include data regarding treatments, medical professionals, assets (e.g., companies, stocks, foundations) or other treatment related output. Individuals who obtain tests about their medical health, genetic markers or genome may want to be able to take action on this medical knowledge. Areas where action may be taken may include: finding treatment methods, finding treatment compounds, finding treatment devices, finding medical professionals for treatment, saving money for treatment, or investing in assets (e.g., companies, non-profits, etc.) which are researching treatments, testing treatments or providing treatments in order to assist with a disease.

Individuals who have a biologic risk profile and/or disease risk profile created for them may allow the biologic risk profile and/or disease risk profile to be submitted to a searching system located in a cloud, on a group of servers or in a local computing system. Disease keywords from these profiles may be used to search through medical research publications, clinical trials, treatment publications, medical professional listings, social media publications, web publications or financial publications in order to identify treatment methods, treatment devices, treatment compounds, treatment related information, medical professionals for treatment, non-profits, companies, or assets to purchase in order to further the development of medical treatments for diseases. The disease keywords may be submitted to machine learning models, matrixes, search indices, graph traversal engines, or other search tools to help identify treatments, research records, test records, assets, medical professionals, medical devices, medical compounds, non-profits, companies or other actionable treatment related items associated with a person's own bioinformation or biological state.

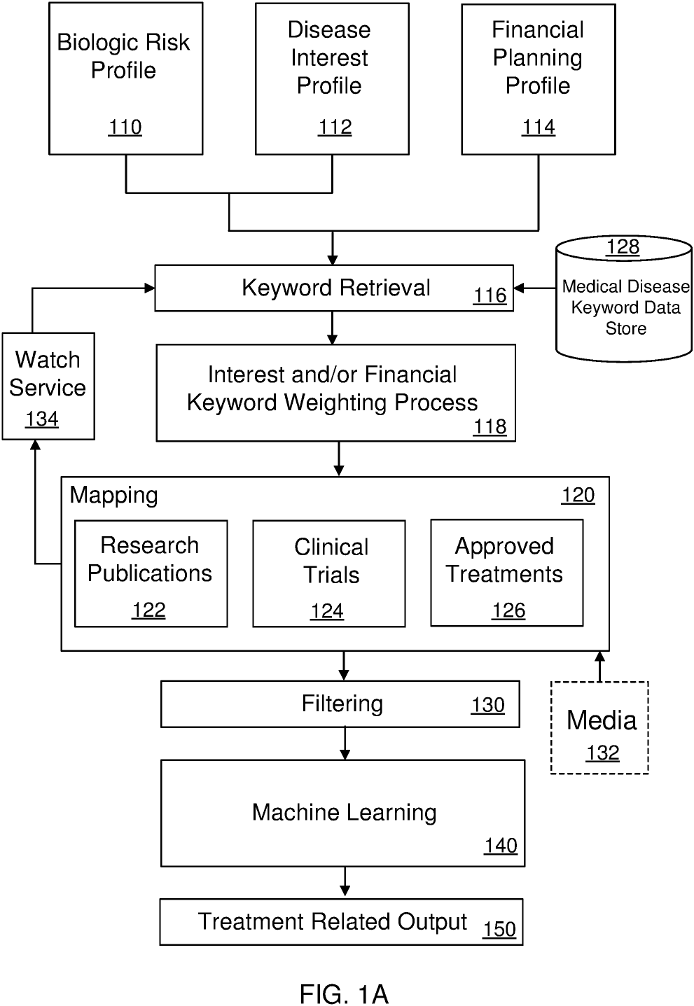

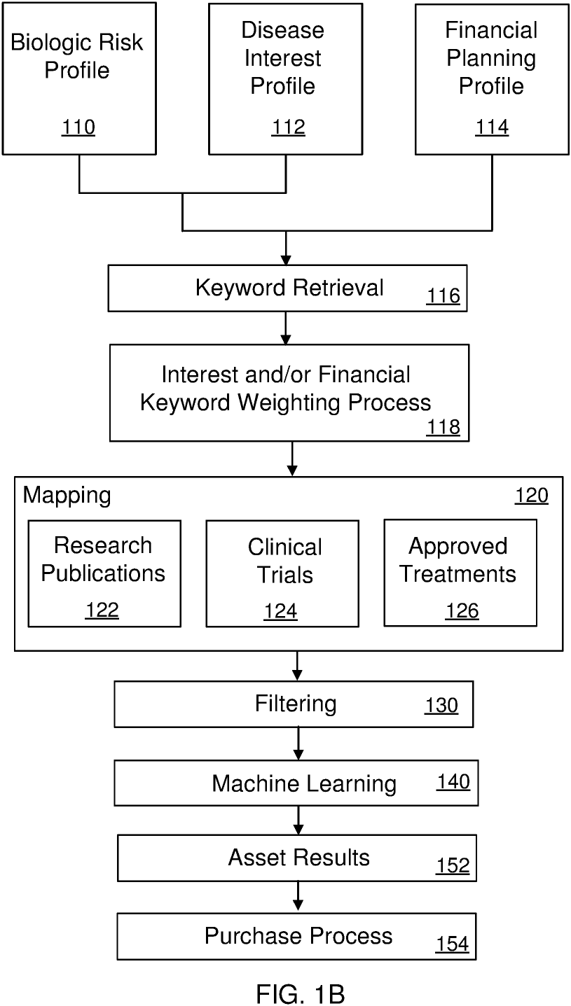

FIG. 1A illustrates that biologic risk profile information about an individual’s biological state may be used to search for treatment related output (e.g., medical results or asset results) which are actionable for the individual. A portion of the individual’s biological state may be represented in a biologic risk profile 110 for a person and may be obtained from a data store. The biologic risk profile 110 may define a plurality of disease risks represented as bioinformatic markers of the person. In addition, the biologic risk profile 110 may include biological information that is specific to the individual’s personal biology and is intended to reveal a personal risk profile for any number of diseases or biological states with respect to disease.

The biologic risk profile 110 may be created by obtaining data that resulted from one or more genetic tests or genetics-related tests. Examples of these genetics related tests may be information from at least one of the following tests: genetic tests, transcriptomics, proteogenomic testing, microbiome tests, or cell analysis. A cell analysis may be obtained using a cell culture based in vitro drug screening. More specifically, the cell culture may include removing cells from an individual and culturing those cells in an in vitro model and then applying a panel of drugs relevant for an associated condition. Another type of cell analysis may be flow cytometry, and flow cytometry may include the identification of immunological signals on cell surfaces from single isolated blood cells. These tests along with any other biological, medical, genetic or genomic information may result in identifying a biological state of the individual that may be captured in the biologic risk profile 110. The biologic risk profile 110 may be stored in a genetic risk data store that is located in a central cloud data store, on a server or on a local computing device.

The biologic risk profile 110 may include bioinformatic markers that are in a machine readable format. These may be bioinformatic markers regarding genes that a person is known to carry as a result of testing. For example, the individual may be known to carry the BRCA1 gene, APEO4 (Alzheimer’s risk) or the Huntington’s disease gene. Another example is that a person’s family may be known to be prone to leukemia, lymphoma or other types of cancer. The bioinformatic markers may represent a person's genetic disposition toward mental illnesses or brain illnesses such as schizophrenia, Alzheimer’s disease, dementia, autism or other diseases related to the brain. In addition, the bioinformatic markers may include functional medical markers such as: a thyroid panel, adrenal stress panel, hormone panel, food sensitivity training, advanced celiac panel, stool analysis, Lyme disease testing or other similar tests. The bioinformatic markers may also be other medical tests such as: blood pressure, blood sugar, blood count, prothrombin time, metabolic panel, lipid panel, thyroid stimulating hormone, Hemoglobin A1C, urinalysis, cultures or other common medical tests that can be recorded in a machine readable format. The bioinformatic markers may include any information known about the person’s genetics, genome or the person’s family genome as identified by family history or in other ways. In addition, the bioinformatic markers may also include data about how a person’s genetics interact with environmental factors (e.g. pollution, substances, etc.).

A disease interest profile 112 may represent diseases in which the person has interests, and the disease interest profile 112 may be obtained from a data store. The data store may be a local data store in a computing device, an on-premises server or a data store located at a cloud service provider. The disease interest profile 112 may include a list of diseases including: diseases afflicting family or friends, diseases related to a person’s profession or business, or diseases related to a personal interest. The disease interest profile 112 may be obtained by surveying the person who owns the biologic risk profile 110 about diseases in which the person is interested. Accordingly, the individual can provide the diseases in which they are personally interested. For example, the individual can list diseases they are interested in by typing and/or selecting diseases or disease types from a list provided to the individual through a user interface. More specifically, the individual may receive an electronic survey in an electronic web application interface or a mobile application interface. Alternatively, the individual or user may be presented with an interface from a process on a computing device (e.g., a mobile device, desktop computer, etc.) that may help direct or guide the individual to conclusions for diseases in which they might be personally interested. These disease suggestions or guidance may be created using demographics known about the individual, area of residence, the biologic risk profile or other factors.

A financial planning profile 114 may also be obtained for the person which can include an amount of risk a person is willing to assume (i.e., risk tolerance), a goal for assets to be accumulated and/or a time frame the person desires to hold the assets purchased (e.g., a time horizon). The financial profile may also contain personal information such as a person’s name, address, age, income or other demographic information. Additionally, the financial planning profile 114 may include goals for the assets to be accumulated, such as: building travel funds, accumulating inheritance funds, accumulating education funds, building a philanthropic foundation, or other outcome goals for the assets to be purchased. The financial planning profile 114 may be used during a subsequent keyword retrieval step to affect the keywords retrieved, and/or the financial planning profile 114 may be used in a later filtering step to filter out mapping or search results that do not match the financial planning profile 114.

Disease keywords may be retrieved or extracted from the biologic risk profile 110 and the disease interest profile 112 using a keyword retrieval process 116 which may reference a medical disease keyword data store 128 and/or other medical term references to determine relevant keywords to be retrieved. The disease keywords may be retrieved from the profiles using automated or semi-automated tools. For example, the system can use existing tools, such as MetaMap, to automate extraction of the keywords from the biologic risk profile 110 and disease interest profile 112 and notate the prevalence and specificity of the keywords. The medical disease keyword data store 128 used by the system may include general medical natural language stems like those from SNOMED or LOINC. The profiles (i.e., reports with biological or genetic testing information) can be provided to the system and may include data from medical partners in formats which may provide disease headings for diseases the individual is currently suffering from or diseases for which the person has an elevated risk. This disease header information may also be used to verify that the correct medical keyword information has been retrieved and/or extracted from the profiles or documents. The disease keywords may be defined to include keywords from one or more of: biological keywords, disease terms, genetic terms, genomic terms, family medical history terms, or medical condition terms.

The disease keywords may be weighted based on the disease interest profile 112 by applying an interest weighting rule using a weighting process 118. For example, the keywords may be weighted based upon the investor’s specific interests. More specifically, the person may have a specific interest in treating a disease the person is genetically prone to develop (e.g., breast cancer) and this disease may be weighted more heavily. In an additional example, an investor may have a specific interest in a particular phenotype of dementia based upon a combination of proteogenomic and family history data that has been identified. In a further example, an investor may have extensive experience working with rare genetic disorders due to the experience of a child or other family member and such disorders may be weighted more heavily in the keywords for the individual.

Accordingly, when there is a greater interest in a disease by the person who owns the biologic risk profile 110, then the disease keywords may be given greater numerical or ranking weight. The weighting may be a number greater than zero, any integer, a floating point number, a probability between zero and one or an alphanumeric grading. In addition, the weighting of the disease keywords may be based on a disease risk of a specific disease for the person who owns the biologic risk profile. For example, if the probability the person has a specific genetic disease is 70% the related disease keywords can be weighted more heavily than if the disease risk is 2%. In the case of a 70% risk probability, the genetic disease may be given a 0.7 weighting.

The keywords may optionally be weighted at this point using financial planning rules. The financial planning rules may be set in the financial planning profile 114 or derived from data in the financial planning profile 114. For example, if the person want a return in five (5) years, then cancer keywords may be weighted less because cancer has historically been more difficult to research or treat.

The resulting disease keywords can be mapped to 120 or searched against research publications 122, clinical trials 124 and approved treatments 126, and the disease keywords can be used to find treatment related output 150. For example, the treatment related output may be assets in which an individual may invest. The disease keywords as mapped 120 using a mapping process or a search process to research publications 122, medical clinical trials 124, or medical treatments 126 can form a medical document mapping. In one configuration, the disease keywords may be indexed to subject headings within reports on public databases. This mapping of disease keywords to medical documents may also be considered an automated developmental assessment because the development progress of treatments may be assessed using this mapping.

Additional medical terms can also be identified from documents in the medical document mapping, and these medical terms can be added back to disease keywords to further expand the searching. The additional medical terms may be selected by a user or machine learning. Additional medical terms that are extracted from the medical disease keyword data store 128, medical term references or medical references may be annotated to show the sources from which the additional medical terms are drawn. These additional medical terms that are found can be combined with the initial keywords (e.g., from the biologic risk profile 110 or disease interest profile 112) to form search terms which may help expand the mapping operations described.

The search hits that are displayed for research publications 122, clinical trials 124 and approved treatments 126 can be annotated with the source location or document name and/or network source address from which the individual results were drawn. For example, an investment recommendation may come from a financial analyst’s document as combined with a medical research publication, and the source of both documents may be shown. In a more specific example, the sources of investment results or medical support results supplied using the enhanced keywords found using the medical and academic literature can be shown as a footer or note in the user interface. In one example, the disease keywords may be individually mapped 120 to multiple research publications 122 or multiple keywords may be mapped to research publications that satisfy defined combinations of disease keywords.

The MeSH (Medical Subject Headings) index may be used to find research publications that match with or satisfy the disease keywords (e.g., disease keywords may be used as a query). The Medical Subject Headings (MeSH) thesaurus is a hierarchically-organized vocabulary produced by the National Library of Medicine. The thesaurus is used for indexing, cataloging, and searching for biomedical and health-related information. MeSH includes the subject headings appearing in MEDLINE/PubMed, the NLM Catalog, and other NLM databases. Tools like MeSH provide an index for the vast majority of known human diseases and disorders, and automated searching can be used to tie the disease keywords to the MESH headings. When new headings become available that are relevant, then the technology can link disease keywords to those new MESH headings with a new mapping or search using the disease keywords. The system may suggest MESH headings and return research papers related to the topic of interest. For any other medical databases with a controlled vocabulary, the technology may build similar indexes, hierarchies or ontologies to connect the keywords retrieved to medical or research publications.

In another example, Google Scholar may be searched using saved search strings or search strings may be submitted to the Google search engines using Google’s search APIs (Application Programming Interfaces). This type of search interface may also run searches built around permutations of disease keywords using search practices defined for the medical fields.

The technology may further weight keywords and/or the results of the searching on the research publications 122, clinical trials 124 and approved treatments 126 based on at least one of the following metrics: a combination of records returned, a number of references found, an impact factor of references, keywords associated with the study that correlate to developmental stage, and/or the presence of commercial authors on the papers. Alternatively, the weighting of the results may be a combination of the disease keyword weighting and the metrics described above in this paragraph.

The technology may use the disease keywords to search through or map to a listings of clinical trials 124. For instance, the disease keywords may be used to search the listings in clinicaltrials.gov, foreign medical trials or private clinical trial information in databases. The results of using the disease keywords may provide results containing relevant diseases or ‘conditions ’for which there is one or more listed clinical trials. Any clinical trial that is terminated or unknown may be filtered from the search results or excluded using the search query terms. However, trials with specific attributes may be prioritized, such as: later stage clinical trials (especially phase 3 and 4 trials), clinical trials with sponsors that include companies listed on public exchanges, or clinical trials with sponsors that are not institutional sponsors (e.g. not universities or research entities).

The technology may also use the disease keywords to query the database of the National Library of Medicine drug information portal or similar approved drug data stores to identify approved treatments 126 products on the market, and/or the companies that sell treatments or devices. The query may result in a listing of products for sale and the companies that sell the products. Companies that are traded on public exchanges may be prioritized. Any other foreign or private data stores that list approved treatments or alternative treatments (e.g., cannabis, Lion’s Mane, etc.) may also be searched.

The approved treatment results may be used directly or weighted according to the medical mapping. In one configuration, the approved treatment results may be weighted according to the medical mapping based in part on at least one of: a number of medical publication occurrences, an impact factor of medical references, developmental stage of a study, commercial authors, existence of a clinical trial, stage of a clinical trial, commercial clinical trials, existence of drug treatments, or commerciality of drug treatments. Alternatively, the mappings may be directly used as features with or without weighting that are recorded in a data store or vector for the person and the features may be submitted to a machine learning model 140 for processing without any weighting. The machine learning model may then provide a classification or output to the user representing a recommended treatment that was be identified using the machine learning. In one more specific example, the medical treatments may be weighted as greater than medical trials or medical publications because the medical treatments have immediate application and the medical trials or medical publications are more speculative.

The integration of the results for the search parameters can provide a snapshot of the current state of research that is relevant to the treatment or curing of each disease in order to formulate suggestions of specific treatments, medical professionals or assets that are related to the disease. Thus, the output can be a road map for assisting with the individual’s health and wellbeing. The system may identify and provide information regarding names of medical specialists who are experts on an identified disease condition. The output of the system may also identify a level of expertise and a location of the doctors or medical professionals who may assist with treating a disease or condition. This provides the individual with the biologic risk profile concrete paths for treating or even curing a specific disease or condition. For example, an electronic page or a tab in the application may be named “Medical Support Search”. When the page or tab is selected, the keywords may be presented for searching. The user may pick which keywords the user want to perform a search with and the sources they want searched using the keywords the user selected. The results can be specific treatment providers with their known expertise, the treatment provider’s location and similar information. The treatment providers may be doctors, hospitals, clinics, medical researchers, companies, or other medical service entities in the United States or internationally.

In one configuration, weighing or prioritization may also occur based on treatment information in the research publications 122, clinical trials 124, or approved treatments 126 (e.g., products). More specifically, the weighting may be based on the furthest developmental effort. The developmental efforts that are identified as being furthest along may have greater weighting applied. Alternatively, treatments that are the furthest along may be weighted less if the treatments are considered to be ineffective and a person may desire to invest in emerging research or emerging treatments instead. Results can also be weighted based on a rule for distributing the investments in various areas. For example, the distribution rule may be to distribute 20% to cancer research, 20% to mental health research, etc. In one example, the recommended assets may just be an option presented to an individual or stock broker and the stock broker or the individual purchasing the assets may have to determine whether an investment is actually made.

While the disease keywords are suited for searching databases of medically oriented data, the same disease keywords can also be used in other automated search protocols or media 132. For example, the disease keywords can be used to search other types of media such as social media or websites for postings or hashtags, medical websites to find relevant new discoveries, or news websites to search for results related to the disease keywords. Examples of social media that can be searched may include: Facebook, YouTube, Instagram, TikTok, Twitter, Pinterest, SnapChat, LinkedIn, Medium, Foursquare, etc. Other media 132 data stores may be searched such as Bloomberg, Thompson Reuters, API, Lexis Nexis, Westlaw and other proprietary platforms, which provide keyword search functionality to find articles or information related to the disease keywords. In some situations, the media reports or online reports may be available before other more formal medical information. Younger researchers and doctors may sometimes release preliminary results on social media or the open internet. In such cases, these social media or web results can be weighted and included in identifying the list of possible assets that might compose a hedge fund of assets.

The weighting of the disease keywords and/or results from searching the research publications 122, clinical trials 124 or approved treatments 126 may also be based on supplemental factors that may include at least one of: a regulatory burden, a regulatory path, a pre-market approval, a predicate approval, a minimal regulatory burden, physician adoption, insurance reimbursement, or investor interest. The supplemental factors may be factors that are more subjective and are based on government regulation, health management organizations (HMOs), medical treatment providers' acceptance stances with respect to research, clinical trials, marketing channels, venture capital or other non-scientific factors.

In an alternative configuration, the documents found in searching or mapping of the research publications 122, clinical trials 124 or approved treatments 126 may be weighted. These documents may then be filtered to remove documents below a desired threshold. Then document keywords may be extracted from the search results and added to the disease keywords for finding treatment related results or assets. In one specific configuration, terms extracted from the documents may be used as direct results provided to a person. For example, a person may receive names of treatments, doctors, companies or other assets directly from the searched documents.

A filtering 130 operation on the keywords may occur. The filtering 130 may select disease keywords with a weighting above a defined weight threshold, and disease keywords below the defined weight threshold may be filtered out during the selection process. For example, some disease keywords may not receive any weighting (e.g., zero weight) because no results have mapped to the disease keywords, or the disease keywords may have a low relative weighting (e.g., a partial reference, an incomplete trial, an ineffective product, etc.). These filtered out keywords may be considered “dormant” but may not be discarded and may be used in later iterations that re-execute the search process.

The filtered out disease keywords may be archived with the other extracted keywords for a repeat search at a later time. The search using the disease keywords may be re-executed periodically, such as every week, month or every quarter. The repeat searches may be performed with or without dormant keywords or the dormant keyword may only be used when searching again after some period of time (e.g., 1 month, 3 months, 6 months, 1 year, 2 years, etc.). The repeated searching is valuable because new discoveries may arise which apply to the disease keywords originally pulled from the profiles. For example, if a newly developed gene editing tool arises, the gene editing tool may apply to disease keywords for which there were previously no results. Similarly, if an unpopular gene editing tool suddenly becomes popular, then references to the gene editing tool in the medical literature may later result in weightings for unused disease keywords. New weightings may also occur based on a study occurring or similar medical progress.

Repeated searches using the disease keywords may be performed using a watcher service 134 in a cloud or a cron job that may exist on server to perform the search. The watcher service 134 can: report on research or treatments as they publish (e.g., PubMed), identify companies as the companies progress through FDA approval, identify reports of clinical trials or identify newly approved treatments, etc. Any identified developments can trigger a new search and weighting cycle. As a further example, a notification may be received that data inputs have changed from one or more of the sources of initial data. These sources of data may be: disease keywords, medical publications, medical trials, medical treatments or other sources of data related to the biologic risk profile. When a notification from the watcher service 134 or watcher agent indicates that one of these sources of data has been updated, then a search to identify at least one treatment related output or asset can be re-executed.

The keywords and weightings may be used in identifying at least one treatment related output 150 that is associated with the disease keywords. A machine learning model 140 may be used to process the disease keywords and respective weightings as features for the machine learning model. The machine learning model may use at least one of the following models: a classifier machine learning model, a regression model, a clustering model, a neural network model, a deep neural network model, a random tree forest model, reinforcement learning, a genetic model, an evolutionary model, natural language processing or any type of machine learning model that can be used for identifying treatment related output.

The machine learning model 140 may identify one or more treatment related outputs 150. For example, any type of machine learning model that is capable of selecting one or more assets, treatments, or medical providers (e.g., treatment related outputs) may be used. These treatment related outputs may be assets that are associated with the biologic risk profile 110 and/or disease interest profile 112. For example, a classifier type of machine learning model may be able to receive features (e.g., disease keywords) from the profiles and classify an individual asset or an asset fund that fits the individual. This type of classification may be performed with regression, random tree forests, neural networks or other classifiers. A fund of assets may cover a group of assets that promote or invest in developing treatments or existing treatments for types of disease risks, such as cancer risk, diabetes, Alzheimer’s disease, mental illness, hemophilia, or heart attack.

Clustering, such as K-means clustering, may also be able to use features from the disease keywords for an individual to find assets for the individual. For example, the features from the profiles and/or search results may be used to create a vector (e.g., vector quantization) and the vector can be used to find similar clusters. A cluster of cases or observations with the nearest mean may be the selected cluster for the incoming profiles and search result features. When the vector is nearest to a specific cluster, then the output for that cluster may be an investment asset or a hedge fund associated with that cluster. The clustering model may be trained by providing a number of training cases or observations that have already matched individuals together with an asset.

Another type of searching that may be used includes graph data stores for mapping or searching through the research publications 122, clinical trials 124, and approved treatments 126. Graph data stores may use a graph structure for semantic queries. Each node in the graph may contain nodes to store the data and edges to connect the nodes based on meanings or relationships between the nodes. The relationships allow data in the store to be linked together directly and, in many cases, retrieved with one operation. Graph databases can store the relationships between data as a priority. Querying may be fast because the relationships are perpetually stored in the database. Relationships can be intuitively visualized using graph databases, making them useful for heavily inter-connected data. These types of databases can be considered NoSQL databases, a Key-Value data stores or document oriented data stores. The relationships between nodes in the database can be used to find related items to the medical keyword being used in the searches. More specifically, connections between nodes may be tested to see if a node may be part of the search results. In this sense, related medical documents that are linked to nodes found using the search keywords may also be determined to be related to the other documents using semantic links. In an additional version of the searching, a visualization graph may be presented to the user and the user may determine how far along the graph the search may proceed.

The machine learning model(s) may be trained using training data representing treatment related output obtained for individuals with a defined listing and weighting of disease keywords. For example, a significant number of labeled training cases (e.g., labeled as having good treatment related outputs) may be provided that list good treatment related outputs for a number of varying biologic risk profiles 110. Thus, when a person with a similar biologic risk profile 110 and/or disease interest profile 112 is submitted to the machine learning model, then similar treatment related outputs may be identified for the new case.

Feedback can also be provided on each treatment related output selected for a person. Any treatment related output that is actually selected by the individual (e.g., asset, doctor, company, treatment, etc. that is selected and is believed to help the disease) may be labeled as a good treatment related output for the features submitted for that input case. Any treatment related output that is marked as being not a good choice for the features which are submitted may be marked as a negative case. These marked feedback cases can be used to re-train the machine learning model being used. This re-training may take place in an offline mode when enough training cases are collected or the re-training may take place immediately, as the feedback is provided.

Similarly, feedback can be used to train a neural network model. When an asset is selected or confirmed for investment by an individual, then this selection can be used to strengthen the weights on inputs or outputs of the neural network or the stages of the neural network. Over time, a neural network may be tuned to determine whether an asset, class of assets or hedge fund may be recommended for an individual with an input biologic risk profile, for example.

Optionally, the machine learning model may also use input features provided by the financial planning profile 114. In one example, the amount of financial risk that is desired may be entered into the machine learning model and the financial risk may be a machine learning feature used to identify at least one treatment related output with an amount of risk that is driven by one or more features of the financial planning profile.

In one configuration, the biologic risk profile 110, disease interest profile 112, and the financial planning profile 114 may be changed or updated as additional biological testing, disease interests or financial preferences are electronically received or are provided by an individual. This means the profiles may be evolving metrics, markers or attributes that can be automatically updated when new information for the individual is received for one or more profile areas. Accordingly, the keywords extracted for the search or mapping process related to the individual can be updated when the biologic risk profile 110, disease interest profile 112, or financial planning profile 114 are modified or updated. For example, when new data comes into a data storage location (e.g., storage bucket) or an update service (e.g., a micro-service) in a computer network that pertains to an individual’s profile(s), a new search or mapping process may be retriggered. This mapping or search may result in identifying treatment related output (e.g., assets). Then trading software may automatically make trades to purchase assets when the individual has given full discretion for making trades. Alternatively, the software may notify a consultant or trader about the new data and allow the consultant to ask the individual if the new data can be acted upon to make an asset acquisition (e.g., a trade). This allows an individual to act on an advancement in health technology physically (e.g., by providing trading instructions, by visiting a recommended doctor, etc.) or by modifying their investment strategy in some way.

FIG. 1B illustrates that the treatment related output 150 of FIG. 1A can be an asset result 152 identified by the machine learning model 140 and process(es). In one example, the asset results 152 identified may be companies providing medical treatments for diseases related to the disease keywords, and the companies are identified using the disease keywords as features submitted by a process to a machine learning model in order to identify companies as output from the machine learning model.

Once one or more asset results 152 are identified, then a purchase process 154 may purchase at least one asset that is a stock or equity in a company that was identified. The purchase process 154 may execute automatically using a public or private exchange or the purchase process may execute upon receiving instructions from the person who owns the biologic risk profile.

In one automated configuration, the purchase process may run in real-time. Real-time is defined as receiving news that a new research publication, clinical trial or treatment is available and then a trading action is performed the same minute, hour or day. The receipt of news documents or input may trigger a disease keyword search using search services in the cloud or an on-premises server. After the search is performed, then the machine learning algorithm may provide a result to recommend an asset, and the trade can happen in real-time. For example, the automated trade may buy a 1% or 5% allocation of a person’s portfolio in the recommended asset. Depending on the configuration, the purchase process 154 may perform: automatic asset purchases, asset sales or asset trades based on owner approval. Alternatively, the purchase process 154 may execute when a broker representing the person who owns the biologic risk profile initiates a purchase or sale. In addition, the assets may be investments in non-profit organizations, investments in research, investments in charitable foundations, charitable gifts, venture capital or private equity funds. In one configuration, sophisticated investors or experienced investors may identify investment thresholds (e.g., dollars to invest) to use for automated trades to invest in any companies that emerge or hit a certain developmental threshold and correspond to a set of disease keywords.

In a further configuration, the machine learning model may be used to identify medical professionals (as opposed to assets) or medical clinics which may be associated with the disease keywords and weightings for the person. Identifying one or more medical professionals or medical clinics who specialize in a particular disease in a defined geographic area may be of particular importance to the person with the biologic risk profile in seeking treatment. Thus, in the event the person needs to seek medical treatment for a specific medical condition identified in the biologic risk profile 110, the individual will have actionable information due to the search performed using the disease keywords.

In another example configuration, the individual may be presented with a series of choices in the asset results 152 that are listed in a graphical user interface. These choices may include research investments, clinical trial investments, product investments or approved treatment investments. Such choices may give investors a broad range of medical investments in which to use their investment resources to advance research for diseases, address symptoms of diseases or cure the medical conditions and diseases that matter to the individual. Individual investments can be presented to the individual and these investments may be assets like an investment in an individual company or individual research fund.

In another configuration, the technology can offer a portfolio of assets, with multiple companies or multiple funds that allow the investor to hedge investments on existing products or developing products. A portfolio of broader investments in the biotechnology sector may be directed, as closely as possible, to the diseases that matter to the investor. For example, a hedge fund of assets may be created based on assets that are actually available as a potential treatment for a disease the person has or is interested in. Furthermore, if there is a company that has a product on the market for a specific disease, or there is a cure for that disease, then the hedge fund or portfolio can be weighted toward those treatments that have greater efficacy. In another example, the only assets available may be an ETF (exchange traded fund) with a company that is doing early stage product work, and then the hedge fund or portfolio can be invested in the company in the ETF based on the just early stage work. If there are no products for treatment being researched, tested or marketed then a person can invest in companies or research groups for biotechnology tools that enable the biotechnology research to occur and which may in the future result in disease treatment products. The machine learning models or the matrix can be used find such companies, as described earlier.

The portfolio recommendations can change over time, based on changing or emerging research and development for diseases. As researchers publish new research publications 122, the technology may reallocate portfolios based on periodic new opportunities in research investment in emerging fields related to the disease keywords of the individual. This means that the disease keywords for the individual may be re-mapped or re-searched in the data stores containing the corpuses of medical information (e.g., research publications 122, clinical trials 124 and approved treatments 126). This may further result in different or additional asset results or medical professionals being recommended to the individual. As new clinical trials 124 commence or advance, individuals can reallocate portfolios to use new developmental investments recommended using the technology. As clinical trials produce newly approved treatments 126 or approved products, the portfolios can be reallocated to make the newly available product investments. The updated portfolio recommendations can also result from re-training and re-applying the machine learning models.

The machine learning models described earlier may further integrate the risks provided by the financial planning profile, which may correspond to the acceptable risk, reward, scope of investment, or timeline for the investment which an individual may desire. Further, automated thresholds may quantify risk, as determined by the scope of the identified furthest developmental effort, the product concept or other multifactor risk assessments. Asset recommendations can be made based on pre-set thresholds for risk. For example, some assets may exceed an individual’s risk tolerance and such assets may be avoided for that individual’s portfolio.

Risk may be further analyzed by using more complex data analytics that factor in the past success of companies in related spaces based on upon the medical area and disease keyword characterization. The past success of companies in related spaces may be used to weight or prioritize the asset recommendations. For example, companies that have been successful in bringing treatments to market may receive greater weighting in recommendation results. Whereas, unknown companies or startups may receive lesser weightings or lesser consideration in the machine learning model. Sophisticated investors can also identify developmental thresholds to use in automated trades to invest in any companies that emerge or hit a certain developmental threshold and correspond to a set of disease keywords or meet other thresholds.

In the past, computing systems have been used to obtain financial information, such as pharmaceutical purchases, to make a medical diagnosis for an individual identified in the computing system. In contrast, the present technology is used to assist in identifying and developing treatments once a biologic risk profile exists for the individual. Thus, this technology can use a marker of disease risk or even a full diagnosis to identify assets that might be purchased. When the individual uses this technology to make investment decisions, then the individual is investing in: 1) their own future, 2) the future of their entire family tree and 3) the future of anyone in the world who may have a similar genetic risk or diagnosed disease.

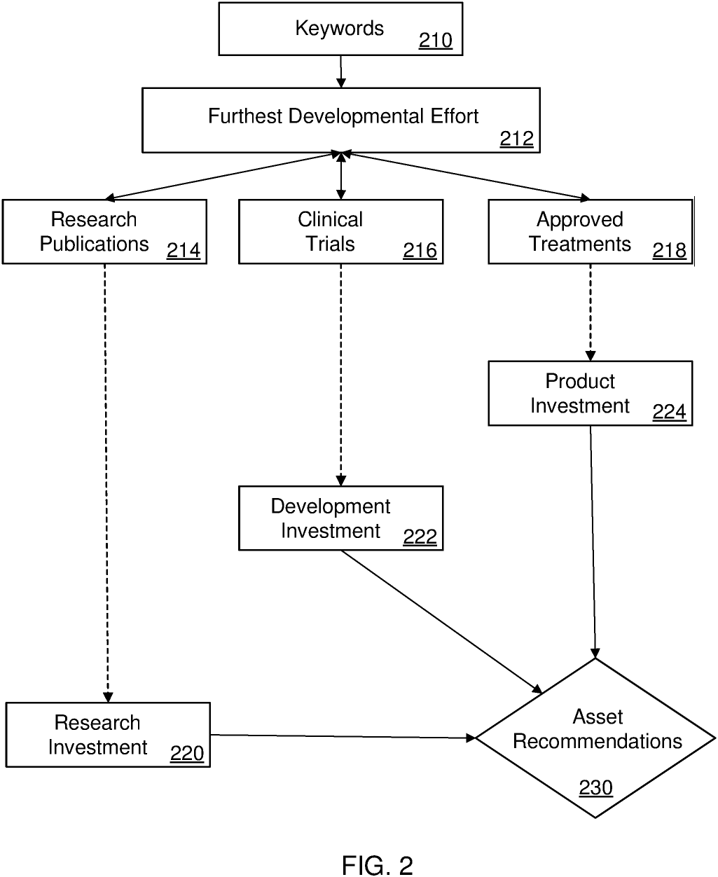

FIG. 2 is a flow chart illustrating identifying an asset that is the furthest developmental effort for a genetic risk and the asset may be recommended for the person. The disease keywords 210 that have been extracted from the biologic risk profile 110 and/or the disease interest profile 112 (FIGS. 1A and 1B) may be used to identify the most mature medical treatment developments that may exist. Accordingly, automated thresholds may quantify risk, as determined by the scope of the identified furthest (i.e., most mature) developmental effort 212, the product concept or other multifactor risk assessments.

The furthest developmental effort may be identified by mapping the keywords to or searching research publications 214, clinical trials 216 and approved treatments 218. The furthest development module may compare the mapped results from each of the sources to determine whether there are results in each area of research publications 214, clinical trials 216 and approved treatments 218. For example, if there is an approved treatment 218, this may result in identifying a product investment 224, which in turn may result in an asset recommendation 230. The asset recommendation 230 may be a stock, bond, a venture capital investment, a private equity investment or another investment in a company that is making the product. Similarly, if there are clinical trials 216 that exist, then the clinical trials may identify a development investment 222 and an asset recommendation 230 for an investment or company that is performing the clinical trials. Further, if there are research publications 214 that exist, then these research publications may identify a research investment 220 and an investment, stock, fund or company associated with the research investment 220 that may be an asset recommendation 230.

An approved treatment 218 may be considered to be a more mature development investment than a clinical trial 216. Similarly, a clinical trial 216 may be considered a more mature development investment than a research publication 214. Thus, the development investment that is most mature may be selected for making the asset recommendation 230. Alternatively, the state of the development of the treatment may be compared to the risk desirable to the individual to determine whether, for example, a developmental effort can be recommended to the individual. The maturity of the investment may also be determined by checking the terms in the documents or electronic records to see if certain progress terms or time terms occur or do not occur in the research publications 214, clinical trials 216 or approved treatments 218.

The asset recommendations 230 for an investment portfolio can come from a machine learning model or matrix which considers the risks of each stage of the developmental effort in light of the financial planning profile to identify asset recommendations. Furthermore, the machine learning model or matrix may identify assets corresponding to an acceptable risk, reward, or scope of investment in a grouping of investment assets. As discussed earlier, the investment recommendations can be made based on pre-set thresholds. Assets that exceed a certain level of risk for a person or investor may be filtered out. Medical diseases that are below a certain level of risk for a person in their biologic risk profile may also be filtered out from consideration.

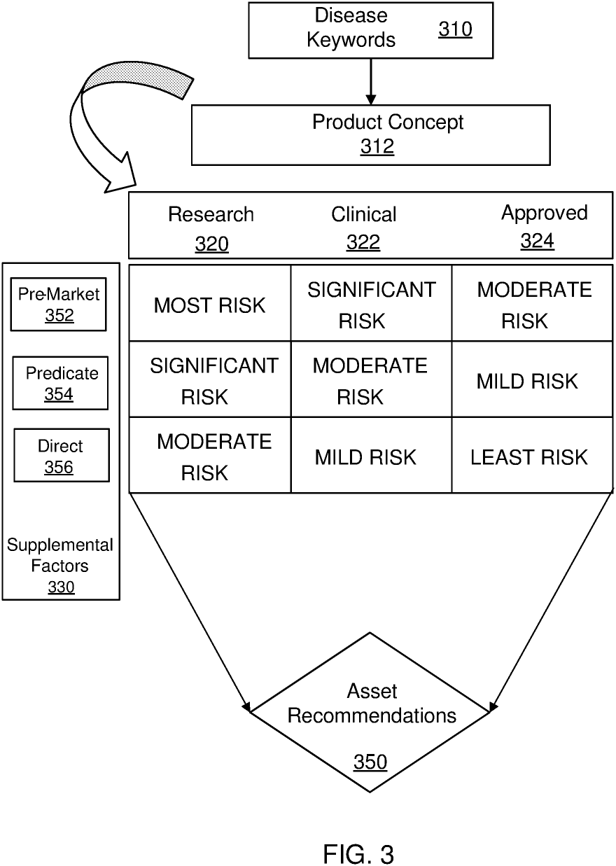

FIG. 3 further illustrates that the disease keywords 310 can be used to identify a product concept 312 that is formed from the disease keywords or using the disease keywords. The product concept 312 may be a collection of terms that represent aspects of a specific disease or aspects of a desired treatment. The product concept 312 can be used integrate multiple development and risk factors in searching for assets and combine those risk factors associated with product development with the available documents identified for research, clinical and approved treatments documents. This combination may also accommodate a supplemental factors 330 dimension and/or financial planning profiles for customers, individuals or investors.

In order to expand the disease keywords 310 into a more product concept 312, the system can further map disease keywords to the terms in the research publications 320, clinical trials 322 or approved product descriptions 324 to establish one or more supplemental factors 330. The product concept can help identify sufficient regulatory, product state, and market information to populate the supplemental factors 330 and integrate those factors into asset purchase decisions.

The supplemental factors 330 can consider multiple product emphases not illustrated, including physician adoption, medical reimbursement rates, or investor interest. The supplemental factors 330 may be combined with the primary research stage analysis 320, 322, 324 to generate matrixes of two or more dimensions (e.g., including multi-dimensional matrixes) to integrate the information into a single assessment. Further, the system can integrate one or more supplemental factors 330 to create multi-dimensional matrixes where each dimension represents a type of supplemental factor that affects the search for assets. The matrix may have any number dimensions that is desired to be considered.

Besides adding another layer of dimensionality to the search or lookup matrix, the supplemental factors 330 can cover attributes that may improve the asset recommendations. For example, the supplemental factors may cover regulatory burden, marketing burden, international market factors, business dimensions, investment dimensions, intellectual property dimensions, legal dimensions or other supplemental factors. These supplemental factors 330 may be independent of the primary factors.

As mentioned, one supplemental factor 330 may be regulatory burden. When the regulatory burden is high then the risk may be considered to be greater and weights applied to keywords and/or search results may be lower. The information contained in research 320, clinical studies 322 or approved product descriptions 324 is also relevant to assess a regulatory path. Such regulatory paths can include pre-market approval 352, which requires first in man studies in order to bring a product to market. Another regulatory path can be predicate approval 354 whereby products can claim as predicate an existing product and show equivalency to the existing product. An additional regulatory path can also include direct 356 products that require minimal regulatory burden before being sold to customers.

In one configuration, the individual may be provided with purchase options in the form of a hedged portfolio of assets. For disease keywords 310 representing diseases for which there is an approved treatment or intervention, the portfolio may prioritize investment in those companies. An approved treatment may constitute the furthest development effort 324 giving investors a clear sense of what the closest applicable treatment is for the disease keywords 310 and indicated disease. The approved treatment 324, if sold by a publicly traded company, can represent a direct product investment and gives the investor the option to support the ongoing sales of an existing treatment or intervention.

For disease keywords 310 representing diseases for which there are emerging products, either in early clinical trials or published results of startup companies, these emerging products may provide general exchange traded investments or other kinds of managed assets to support companies that are developing such new treatments. A development investment gives investors access to new companies that may be harder to trade publicly but are engaging in compelling work. Such companies may be identified by research publications 320 or by clinical trials 322 and may be part of broader managed investment products. Alternatively, broader, industry-wide managed investment products can serve to generally support a specific sector of emerging companies (e.g., gene editing) rather than a general technological sector comprising several large companies (e.g., biotech).

For disease keywords 310 representing diseases for which there are no emerging products but there is ongoing research, the technology may identify or establish general index funds comprising biotechnology companies working in relevant fields building relevant technologies that will be the basis of future products. A research investment may support the companies building the tools to lay the foundation for future products. Those research companies can be identified based on disease keywords 310 associated with the relevant diseases and the technologies enabling further investigation into the relevant disease mechanisms.

For disease keywords 310 for which there are no identifiable products but there are ongoing clinical trials 322 or research publications 320 that are associated with the disease keyword 310, the technology may also contain research tools used to investigate the underlying biology of the condition behind the disease keywords. The system may also recommend ways to invest in those companies that enable research or otherwise make the products to advance that research.

A matrix can assemble these categories and integrate the stage of the investment as well as the potential risk. For instance, a research publications 320 that describes a product requiring pre-market 352 government approval is an investment with substantial risk due to the product’s early stage and long path to market. Similarly, the regulatory burden can serve as a proxy for other interests (e.g., alternative medical treatments), where the system identifies opportunities in dietary supplements or natural extracts as direct 356 products due to their lack of regulatory requirement. The matrixes allow for portfolio recommendations ensuring that the biologic risk profile 110 (See FIG. 1) (i.e., individual’s biological state) and disease interest profile 112 recommend assets that satisfy the investor’s financial planning profile 114. Disease keywords or mapping results that fall in area of risk that are unacceptable to the individual may be filtered out before assets are recommended.

The features and supplemental factors illustrated as being in a matrix can also be processed using machine learning, as described earlier. For example, the features (e.g., keywords), supplemental factors and/or mapping results may be used as inputs to a neural network. The output of the neural network may be one or more assets or portfolios based on the multiple inputs to the neural network.

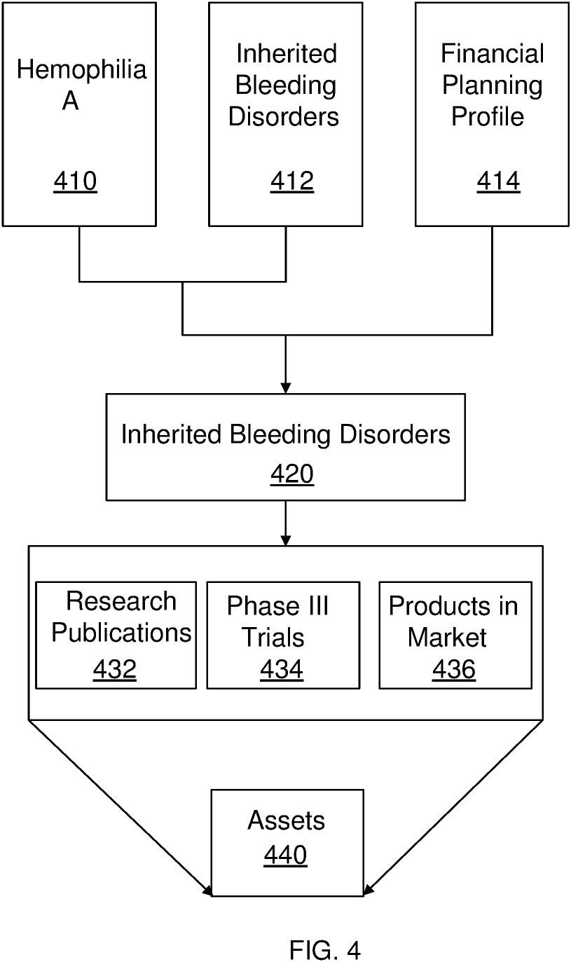

FIG. 4 illustrates the use of the present searching system as applied to the specific bioinformatic marker of hemophilia. The system can retrieve the bioinformatic markers from the person’s biologic risk profile. In this example, the bioinformatic markers may indicate that the individual has risks for Hemophilia A 410. In addition, the disease interest profile may contain data that indicates that the individual is interested in inherited bleeding disorders 412 and skin cancer. Further, information may be identified regarding the individual’s financial planning profile 414.

Keyword extraction may then take place that indicates that the individual is primarily searching for and is interested in inherited bleeding disorders 420. These keywords may then be mapped to data stores representing research publications 432, trials 434, or products 436 for treatment. For example the disease keywords may be mapped to research publications 432 using the MeSH subheadings. The disease keywords may be mapped to clinical trials to identify phase II clinical trials 434. In addition, the disease keywords may also be mapped to medical products in the market 436, such as pharmaceutical treatments, biological treatments, medical devices, etc. The disease keywords may be weighted based on the items found in the search or the items that are mapped to. Then the weighted keywords and/or mapped results can be used to identify assets 440 using machine learning or matrixes.

The ratio of the assets that may be acquired by the person with the bioinformatic markers may also be defined. For example, the individual may decide to weight 10% of their portfolio toward assets that support research for hemophilia, 20% of the assets toward companies with treatments undergoing clinical trials for hemophilia and 30% for products in the market for hemophilia, while the remaining 50% of their portfolio may be invested in index funds or other general investment products.

In the example of hemophilia, there may be many tools that could eventually assist with treating or curing hemophilia. Some of these forward looking tools that might be invested in by the individual and can include: gene editing, gene therapy, biologics treatments, software tools, devices or any type of tool that might assist with hemophilia treatment development. More specifically, the gene editing technique CRISPR has recently been used more effectively and may be considered relevant to the future treatment of many genetic disorders. Thus, investment in CRISPR like techniques or tools may be selected for individuals who have keywords that align with selecting CRISPR. As another example for hemophilia, there might be early stage hemophilia treatments that might be under consideration or there may be hemophilia clinical studies underway. When these treatments emerge or become approved, the treatments or products may allow the individual have a constantly adjusted portfolio based on the research papers, clinical trials or treatments that develop (or may be dropped).

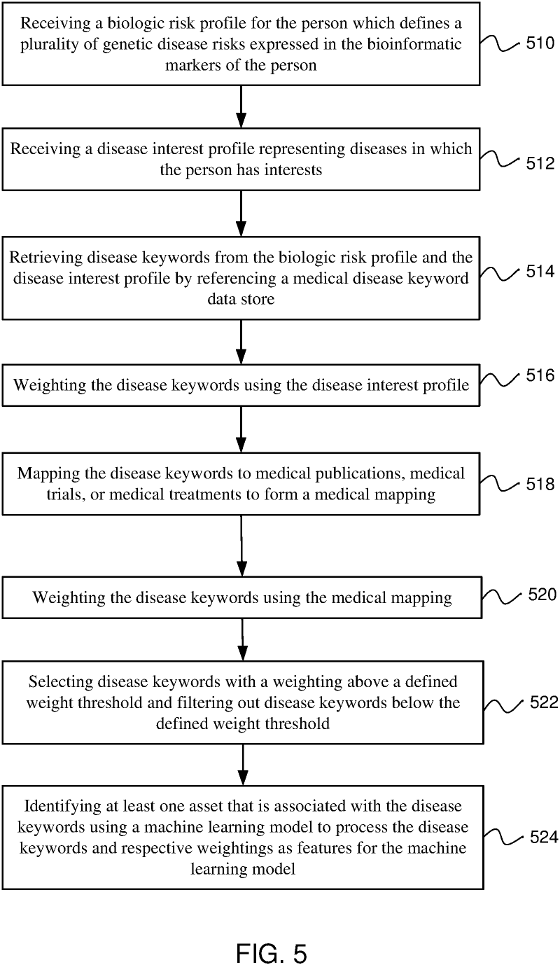

FIG. 5 is a flowchart illustrating a method for searching for assets or treatments using a person’s bioinformatic markers. An initial operation is receiving a biologic risk profile for the person, as in block 510. For example, the data in the biologic risk profile may include information from: genetic tests, transcriptomics, proteogenomic testing, functional medicine tests, microbiome tests, cell analysis, or any other medical, genetic or biological test that may be performed on an individual. The biologic risk profile may define a plurality of disease risks expressed by the bioinformatic markers of the person.

A disease interest profile may be received that represents diseases in which the person has interests, as in block 512. The disease interests may be a listing of diseases that are relevant personally to the investor without necessarily being part of the investor’s unique biology. As discussed earlier, the disease interests maybe diseases afflicting family or friends, diseases related to an investor’s profession or business, or diseases related to any other personal interest of the investor.

Another operation in the method may be retrieving disease keywords from the biologic risk profile and the disease interest profile by referencing a medical disease keyword data store, as in block 514. The medical disease keyword data store may be as simple as a listing of medical terms in a flat file or a more complex data store, such as: an XML file, a NoSQL data store, a relational database, object-oriented database, a graph database, an ontological database, or any other data storage and retrieval technique for storing medical terms in an organized fashion.

The disease keywords may also be weighted using the disease interest profile, as in block 516. Optionally, some disease keywords may be filtered out at this point if the weightings of the disease keywords are deemed to be high enough.

The disease keywords may be mapped to medical publications, medical trials, or medical treatments to form a medical mapping as in block 518. This mapping may be a one-to-many mapping where one keyword maps to many publications, trials or treatments. In another configuration, the mapping may be many-to-many where each keyword maps to many documents and each document maps to many keywords. The disease keywords and/or mapping results may be weighted according to the medical mapping, as in block 520. For example, the disease keywords may be weighted using treatment progress rules that weight results for the documents as greater when the results represent further progress toward a successful treatment. As a result, greater significance may be applied to documents that represent progress that is closer to a successful treatment. In a specific case, with early research, such early stage documents may not be included in the search results. In the case of an effective and mature treatment with a publicly traded company, this document may be included in the search results.

In one configuration, additional terms may also be extracted from mapped documents or the documents found in the searches of the research publications, the clinical trials or the approved products. The terms may be added to the disease keywords and may be submitted to the machine learning or matrixes. These terms may include medical provider names, company names, fund names or other asset results.

The disease keywords that may be used to identify assets may be selected when the weighting of the disease keyword is above a defined weight threshold, as in block 522. Disease keywords below the defined weight threshold may be filtered out and not used in identifying any asset(s). These filtered out keywords may be stored for future reference in the event the search is re-executed.

At least one asset that is associated with the disease keywords may be identified using a machine learning model or matrixes to process the disease keywords and respective weightings as features for the machine learning model, as in block 524. These assets may be assets that are: stock in a publicly traded company, stock in an exchange traded fund (EFT), stock in an investment fund, investment in a venture capital fund, investment in a private equity fund, a charitable contribution (e.g., for tax purposes), ownership in a foundation and any other asset or contribution that may be available. Other assets that may be output by a machine learning model or matrixes may be listings of doctors who treat specific diseases or disease treatments.

The present technology may also be combined with algorithmic trading which uses a computer program that follows a defined set of instructions to place a trade. Once the appropriate treatments, treatment developments or assets have been identified using an algorithm, then the company, stock or other transferrable asset associated with the treatment progression may be purchased.

The defined sets of trading instructions may be based on the treatment state (e.g., most mature treatment) that is desired to be invested in, along with the timing, price, quantity, or defined mathematical model. For example, the trading process may follow simple trade criteria for a desired asset: buy 10 shares of a desired medical stock when a person has a disease keyword related to the stock and the stock’s 60-day moving average goes below the 200-day moving average. Further, the trade process can sell shares of the same medical stock when its 50-day moving average goes above the 200-day moving average. Thus, the trading process can use the medical stocks selected using the disease keywords, the processes described earlier and algorithmic trading to automatically monitor the stock price (and the moving average indicators) and place the buy and sell orders when defined conditions are met. The person, owner or trader may have less need to identify the right stock or monitor live prices and graphs or put in the orders manually. This improved algorithmic trading system finds the stock related to the bioinformatic markers of the individual and performs the trading automatically by correctly identifying the trading opportunity and timing.

The genetic testing that is performed about an investor or user and is used to generate medical keywords or search keywords may be performed anonymously. The anonymous results may be linked to an investment account of an individual by a numbered genetic testing account. Accordingly, the individual being DNA tested may set up an anonymous testing account and send information for the numbered genetic testing account to parties managing investment software and the individual’s investment account. The results of the anonymous DNA test may then be used to determine how to find emerging treatments, treatment providers or investments for the individual or investor. Further, the party being DNA tested may provide approval that their DNA information can be used for medical research but the data may be released for medical research as completely anonymous data.

As a result of anonymous testing, the individual being genetically tested or DNA tested does not need to be concerned that details about their personal genetic background may be revealed to other parties through direct sales of their personal genetic information or that their genetic background may be revealed when genetic test companies are bought and sold. In addition, anonymous testing can avoid situations where the genetic testing data is hacked or leaked on the internet, as sometimes occurs with personal information.

Similarly, the financial investment account may also be kept anonymous and may only be a numbered financial account. In this way, anonymous information about the genetic information may not be linked to the individual through personally identifiable information (PII) associated with the financial investment account.

In a further configuration, the anonymous financial investment account may be used to buy or sell genetically related investments using a block chain that has been created specifically for the transfer of genetically related investments. The anonymous financial investment account that is used to buy and sell genetically related stocks, company shares, bonds, etc. may be entered into the block chain (e.g., as only a number). Then the only person who knows the PII (personally identifiable information) for the investor account is the investor themselves. In one example, this personal information can reside on the individual’s own computer with the financial investment account number and in no other location. Of course, the software that is storing the personal information for the anonymous financial account and/or the anonymous genetic testing may have a feature to back up the information to a separate local storage device or to an encrypted personal cloud account to which no other parties have access.

In one example, a financial investment client may setup a personal cloud account and the client may be the only person that has access to the financial investment account. The financial investment account may be known externally only using a numerical, alpha-numerical, an encryption key or an anonymous alias. This financial investment account may be in an encrypted cloud account. Then an anonymous DNA test may be done and the DNA information may also be stored in the encrypted or protected cloud account. Next, the user may sign up to use their DNA results to search for related investments using their protected account name (e.g., a number or key). The investment software may provide the results for the suggested investments back to the protected account or the investment purchases may be made using the protected account identifier. In one situation, the investment company providing the trading service could also be the entity paying for the DNA testing that is anonymous. The protected investment account may also have an avatar of the investor that has been created. This avatar may be associated with the financial information about the individual but may not have the persons PII or may not make that available. In addition, the avatar may be used to submit an application for a loan, apply for a credit card, or perform other financial transactions using the sanitized financial information about the client.

In one example configuration, the specialized cloud may be set up with templates that only allow information to be passed outside of the cloud storage area if the information matches the template. Thus, templates may be created for passing DNA data back and forth to entities outside the cloud or cloud account, but the templates may not allow for passing personally identifiable information (PII) with the DNA data because the templates do not allow the personal data to be sent outside the protected cloud. Similarly, the investment search or purchase and sale transaction data that is sent or returned from the protected cloud may be transferred using a cloud template and that template will not allow the personally identifiable information (PII) to be transferred but only a protected account name or alias will be used. The cloud templates may block the transfer of any personal information related to the personal DNA information or personal financial information using a specialized hardware and cloud configuration.

In another configuration, the protected cloud account can be hosted by the investment firm or entity and the person owning the investments may be the only one that can access the PII related to the financial information or the DNA test results obtained from anonymous testing. This way the investment firm can protect investor‘s identity while providing access to purchase of the types of medical investments in which the investor has interest. In this configuration, the avatar may be located at the financial institution and then the avatar for an investor may be used for the DNA transactions, financial searching, and purchase transactions. The goal is to have all of an investor’s financial data in a protected form but represented by the avatar. For example, an investor may be able to scan a QR code or provide another code. The scanning or reading of the code may result in uploading information about the financial investor to a financial institution, hospital, car dealership, but the avatar or numbered account protects the PII and provides the financial data without any PII. Having a QR code or another code for obtaining the investor’s avatar allows a financial service provider to perform risk analysis for the investor, provide estate planning, invest based on DNA and perform similar functions without revealing PII. Similarly, an API integration may be provided where information can be accessed from the protected investment cloud account with the DNA information but PII is not provided.

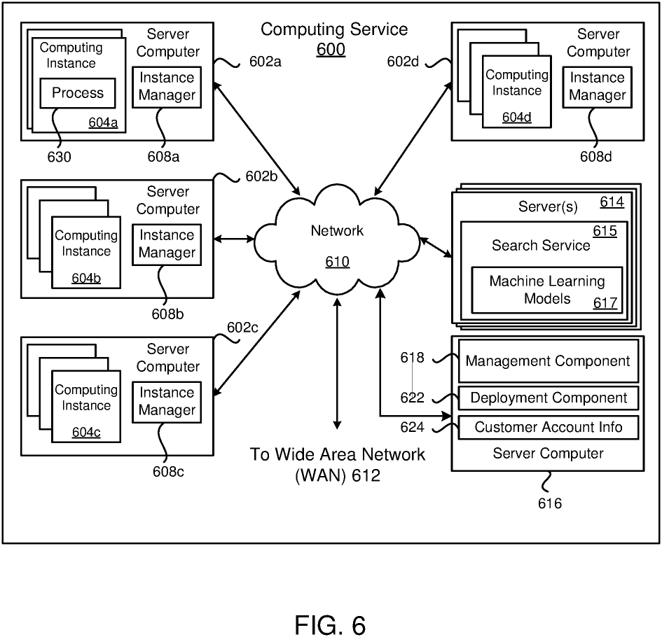

FIG. 6 is a block diagram illustrating an example computing service 600 that may be used to execute and manage a number of computing instances 604a-d upon which the present technology may execute. In particular, the computing service 600 depicted illustrates one environment in which the technology described herein may be used. The computing service 600 may be one type of environment that includes various virtualized service resources that may be used, for instance, to host computing instances 604a-d.

The computing service 600 may be capable of delivery of computing, storage and networking capacity as a software service to a community of end recipients. In one example, the computing service 600 may be established for an organization by or on behalf of the organization. That is, the computing service 600 may offer a “private cloud environment.” In another example, the computing service 600 may support a multi-tenant environment, wherein a plurality of customers may operate independently (i.e., a public cloud environment). Generally speaking, the computing service 600 may provide the following models: Infrastructure as a Service (“IaaS”) and/or Software as a Service (“SaaS”). Other models may be provided. For the IaaS model, the computing service 600 may offer computers as physical or virtual machines and other resources. The virtual machines may be run as guests by a hypervisor, as described further below.

Application developers may develop and run their software solutions on the computing service system without incurring the cost of buying and managing the underlying hardware and software. The SaaS model allows installation and operation of application software in the computing service 600. End customers may access the computing service 600 using networked client devices, such as desktop computers, laptops, tablets, smartphones, etc. running web browsers or other lightweight client applications, for example. Those familiar with the art will recognize that the computing service 600 may be described as a “cloud” environment.

The particularly illustrated computing service may include a plurality of server computers 602a-d. The server computers 602a-d may also be known as physical hosts. While four server computers are shown, any number may be used, and large data centers may include thousands of server computers. The computing service 600 may provide computing resources for executing computing instances 604a-d. Computing instances 604a-d may, for example, be virtual machines. A virtual machine may be an instance of a software implementation of a machine (i.e., a computer) that executes applications like a physical machine. In the example of a virtual machine, each of the server computers 602a-d may be configured to execute an instance manager 608a-d capable of executing the instances. The instance manager 608a-d may be a hypervisor, virtual machine manager (VMM), or another type of program configured to enable the execution of multiple computing instances 604a-d on a single server. Additionally, each of the computing instances 604a-d may be configured to execute one or more processes 630 or applications.

A server 614 may be reserved to execute software components for implementing the present technology. For example, the server 614 or computing instance may include a search service 615 for using the disease keywords to find assets, treatments or treatment providers, and one or more machine learning models 617 to process the disease keywords.

A server computer 616 may execute a management component 618. A customer may access the management component 618 to configure various aspects of the operation of the computing instances 604a-d purchased by a customer. For example, the customer may setup computing instances 604a-d and make changes to the configuration of the computing instances 604a-d.

A deployment component 622 may be used to assist customers in the deployment of computing instances 604a-d. The deployment component 622 may have access to account information associated with the computing instances 604a-d, such as the name of an owner of the account, credit card information, country of the owner, etc. The deployment component 622 may receive a configuration from a customer that includes data describing how computing instances 604a-d may be configured. For example, the configuration may include an operating system, provide one or more applications to be installed in computing instances 604a-d, provide scripts and/or other types of code to be executed for configuring computing instances 604a-d, provide cache logic specifying how an application cache is to be prepared, and other types of information. The deployment component 622 may utilize the customer-provided configuration and cache logic to configure, prime, and launch computing instances 604a-d. The configuration, cache logic, and other information may be specified by a customer accessing the management component 618 or by providing this information directly to the deployment component 622.

Customer account information 624 may include any desired information associated with a customer of the multi-tenant environment. For example, the customer account information may include a unique identifier for a customer, a customer address, billing information, licensing information, customization parameters for launching instances, scheduling information, etc. As described above, the customer account information 624 may also include security information used in encryption of asynchronous responses to API requests. By “asynchronous” it is meant that the API response may be made at any time after the initial request and with a different network connection.

A network 610 may be utilized to interconnect the computing service 600 and the server computers 602a-d, 616. The network 610 may be a local area network (LAN) and may be connected to a Wide Area Network (WAN) 612 or the Internet, so that end customers may access the computing service 600. In addition, the network 610 may include a virtual network overlaid on the physical network to provide communications between the servers 602a-d. The network topology illustrated in FIG. 6 has been simplified, as many more networks and networking devices may be utilized to interconnect the various computing systems disclosed herein.

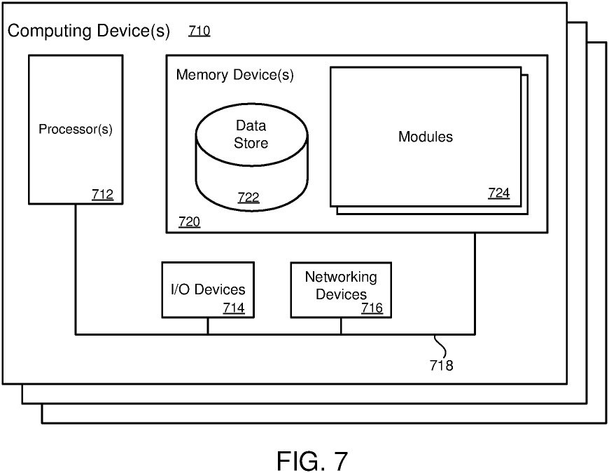

FIG. 7 illustrates a computing device 710 which may execute the foregoing subsystems of this technology. The computing device 710 and the components of the computing device 710 described herein may correspond to the servers and/or client devices described above. The computing device 710 is illustrated on which a high-level example of the technology may be executed. The computing device 710 may include one or more processors 712 that are in communication with memory devices 720. The computing device may include a local communication interface 718 for the components in the computing device. For example, the local communication interface may be a local data bus and/or any related address or control busses as may be desired.

The memory device 720 may contain modules 724 that are executable by the processor(s) 712 and data for the modules 724. For example, the memory device 720 may include an inflight interactive system module, an offerings subsystem module, a passenger profile subsystem module, and other modules. The modules 724 may execute the functions described earlier. A data store 722 may also be located in the memory device 720 for storing data related to the modules 724 and other applications along with an operating system that is executable by the processor(s) 712.

Other applications may also be stored in the memory device 720 and may be executable by the processor(s) 712. Components or modules discussed in this description that may be implemented in the form of software using high programming level languages that are compiled, interpreted or executed using a hybrid of the methods.

The computing device may also have access to I/O (input/output) devices 714 that are usable by the computing devices. An example of an I/O device is a display screen that is available to display output from the computing devices. Other known I/O device may be used with the computing device as desired. Networking devices 716 and similar communication devices may be included in the computing device. The networking devices 716 may be wired or wireless networking devices that connect to the internet, a LAN, WAN, or other computing network.

The components or modules that are shown as being stored in the memory device 720 may be executed by the processor 712. The term “executable” may mean a program file that is in a form that may be executed by a processor 712. For example, a program in a higher-level language may be compiled into machine code in a format that may be loaded into a random-access portion of the memory device 720 and executed by the processor 712, or source code may be loaded by another executable program and interpreted to generate instructions in a random-access portion of the memory to be executed by a processor. The executable program may be stored in any portion or component of the memory device 720. For example, the memory device 720 may be random access memory (RAM), read only memory (ROM), flash memory, a solid-state drive, memory card, a hard drive, optical disk, floppy disk, magnetic tape, or any other memory components.

The processor 712 may represent multiple processors and the memory 720 may represent multiple memory units that operate in parallel to the processing circuits. This may provide parallel processing channels for the processes and data in the system. The local interface 718 may be used as a network to facilitate communication between any of the multiple processors and multiple memories. The local interface 718 may use additional systems designed for coordinating communication such as load balancing, bulk data transfer, and similar systems.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices. The modules may be passive or active, including agents operable to perform desired functions.

The technology described here can also be stored on a computer readable storage medium that includes volatile and non-volatile, removable and non-removable media implemented with any technology for the storage of information such as computer readable instructions, data structures, program modules, or other data. Computer readable storage media include, but is not limited to, RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which can be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared, and other wireless media. The term computer readable media as used herein includes communication media.

Reference was made to the examples illustrated in the drawings, and specific language was used herein to describe the same. It will nevertheless be understood that no limitation of the scope of the technology is thereby intended. Alterations and further modifications of the features illustrated herein, and additional applications of the examples as illustrated herein, which would occur to one skilled in the relevant art and having possession of this disclosure, are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

What is claimed is:

1. A method of searching for output defined by bioinformatic markers of a person, comprising:
receiving a biologic risk profile for the person which defines a plurality of disease risks expressed in the bioinformatic markers of the person;
receiving a disease interest profile representing diseases in which the person has interests;
retrieving disease keywords from the biologic risk profile and the disease interest profile by referencing a medical disease keyword data store;
weighting the disease keywords using the disease interest profile;
mapping the disease keywords to medical publications, medical trials, or medical treatments to form a medical mapping;
weighting the disease keywords using treatment progress rules according to the medical mapping;
selecting disease keywords with a weighting above a defined weight threshold; and
identifying at least one asset that is associated with the disease keywords using a machine learning model to process the disease keywords and respective weightings as features for the machine learning model.

2. The method as in claim 1, wherein weighting the disease keywords according to the medical mapping further comprises weighting the disease keywords based in part on at least one of: a number of medical publication occurrences, an impact factor of medical references, developmental stage of a study, commercial authors, existence of a clinical trial, stage of a clinical trial, commercial clinical trials, existence of drug treatments, or commerciality of drug treatments.

3. The method as in claim 1, wherein the machine learning model is at least one of a classifier machine learning model, a regression model, a clustering model, a neural network model, or a random tree forest model.

4. The method as in claim 1, further comprising:
obtaining a financial planning profile for the person which defines an amount of risk, a financial monetary goal and a time frame the person desires for investments; and
identifying at least one asset with an amount of risk that approximates the financial planning profile.

5. The method as in claim 1, further comprising weighting medical treatments as greater than medical trials or medical publications.

6. The method as in claim 1, further comprising training the machine learning model using training data representing assets obtained for individuals with a similar listing and weighting of disease keywords.

7. The method as in claim 1, further comprising storing the biologic risk profile in a data store, wherein the biologic risk profile includes genetic information from at least one of:
transcriptomics, proteogenomic testing, functional medicine tests, or microbiome tests.

8. The method as in claim 1, further comprising storing the disease interest profile in a disease interest data store, wherein the disease interest data store includes a list of diseases from at least one of: diseases afflicting family or friends, diseases related to an investor’s profession or business, or diseases related to a personal interest of an investor.

9. The method as in claim 1, further comprising identifying assets that are companies providing medical treatments for disease keywords by using the disease keywords as features submitted to a machine learning model in order to identify companies associated with the disease keywords.

10. The method as in claim 9, further comprising executing a trading process to purchase at least one asset that is stock in the companies identified.

11. The method as in claim 1, further comprising assets that are investments in non-profit organizations, investments in research, investments in charitable foundations or charitable gifts.

12. The method as in claim 1, further comprising identifying medical professionals using the machine learning model which may be associated with the disease keywords and weightings for the person.

13. The method as in claim 1, further comprising weighting disease keywords based on supplemental factors that are at least one of: regulatory burden, regulatory path, pre-market approval, predicate approval, minimal regulatory burden, physician adoption, insurance reimbursement, or investor interest.

14. The method as in claim 1, further comprising:
receiving a notification that data inputs have changed for at least one of: disease keywords, medical publications, medical trials, or medical treatments; and
re-executing a search to identify at least one asset.

15. The method as in claim 1, further comprising weighting the disease keywords using a disease risk to the person.

16. A system for searching for assets related to bioinformatic markers of a person, comprising:
at least one processor;
a memory device including instructions that, when executed by the at least one processor, cause the system to:
receive a biologic risk profile for a person which defines a plurality of disease risks for the bioinformatic markers of the person;
receive a disease interest profile representing diseases in which the person has interests;
retrieve disease keywords from the biologic risk profile by referencing a medical disease keyword data store;
weight the disease keywords using the disease interest profile;
map the disease keywords to medical publications, medical trials, or medical treatments to form a medical mapping;
weight the disease keywords according to medical mappings;
select disease keywords with a weighting above a defined weight threshold and filtering out disease keywords below the defined weight threshold;
obtaining a financial planning profile for the person which defines an amount of risk, a financial monetary goal and a time frame the person desires for investments;
identify at least one asset that is associated with the disease keywords by processing the disease keywords using a mapping process; and
filtering out at least one asset with an amount of risk that does not match the financial planning profile.

17. A system as in claim 16, wherein at least one asset is identified using the disease keywords with weightings as entries in a matrix to match assets to disease keywords with weightings.

18. The system as in claim 16, further comprising storing the biologic risk profile in a data store, wherein the biologic risk profile includes genetic information from at least one of:
transcriptomics, proteogenomic testing, functional medicine tests, or microbiome tests.

19. A non-transitory machine readable storage medium for searching for assets related to a person’s genome including instructions embodied thereon, wherein the instructions, when executed by at least one processor:
receiving a biologic risk profile for a person which defines a plurality of disease risks for the person’s genome;
receiving a disease interest profile representing diseases in which the person has interest;
retrieving disease keywords from the biologic risk profile by referencing a medical disease keyword data store;
weighting the disease keywords using the disease interest profile;
mapping the disease keywords to medical publications, medical trials, or medical treatments to form a medical mapping;
weighting the disease keywords according to medical mappings;
selecting disease keywords with a weighting above a defined weight threshold and filtering out disease keywords below the defined weight threshold; and
identifying at least one asset that is associated with the disease keywords by matching the disease keywords with weightings to assets that may be purchased by the person.

20. A non-transitory machine readable storage medium as in claim 19, further comprising identifying at least one asset using machine learning or mapping.
